(19) United States
(12) Patent Application Publication
(10) Pub. No.: US 2020/0046428 A1
(43) Pub. Date: Feb. 13, 2020

(54) FIBRE OPTIC ASSEMBLY

(71) Applicant: Lazcath Pty Ltd, Mosman Park (AU)

(72) Inventors: Peter Pratten, Mosman Park (AU); Kamal Alameh, Rivervale (AU)

(73) Assignee: Lazcath Pty Ltd, Mosman Park (AU)

(21) Appl. No.: 16/343,064

(22) PCT Filed: Oct. 23, 2017
(86) PCT No.: PCT/AU2017/051160

§ 371 Date: Apr. 18, 2019

(30) Foreign Application Priority Data
Oct. 21, 2016 (AU) 2016904283

(51) Int. Cl. A61B 18/24; A61B 5/00

(52) U.S. Cl. CPC A61B 18/24; A61B 2018/00791; A61B 5/0084; A61B 5/0066

(57) ABSTRACT

An optical fibre assembly (10) disposed within a catheter (12) for ablating mammalian, such as heart tissue. The optical fibre assembly has a plurality of optical cores (16A-16C), each core defining a leading end and a trailing end and being adapted to carry an optical imaging beam. An optical lens arrangement (25) is operatively connected to the leading end of the plurality of optical cores for causing divergence of the light beams emitted therefrom. The optical fibre assembly (10) creates a field of view by directing a plurality of said optical imaging beams onto a tissue portion and capturing a reflected portion of said beams. The divergence of the beams provides a greater field of view than may otherwise be provided.

### TECHNICAL FIELD

A fibre optic assembly, an OCT system including the fibre optic assembly and a method of visualising ablation of mammalian tissue in vivo in real time are disclosed.

### BACKGROUND ART

Cardiovascular disease may account for around 30% of deaths in some regions, half of which may be due to heart failure, e.g., progressive alteration of cardiac contraction, which is entirely dependent on prior electrical activation. A substantial number of cases of heart failure are secondary to or aggravated by electrical dysfunctions: e.g., uncoordinated contraction (mechanical dyssynchrony) and heart arrhythmias, the most frequent of which is atrial fibrillation (AF).

Interventionist cardiac electro-physiologists (CPEs) can use minimally invasive thin hollow flexible catheters that are each equipped with a radio-frequency (RF) heater for tissue ablation and an electronic sensor that detects contact between blood vessel walls and the catheter tip assembly. Key advantages of using catheters over open surgery include: faster recovery of patients; less operative time; less morbidity and mortality; and lower cost. Current CPE techniques may also include insertion of a separate ultrasound catheter to determine the thickness of intra-body tissue, e.g., after ablation.

However, current CPE catheter techniques may impose problematic limitations on catheter-based treatment. In particular, the less than ideal visualisation of the ablation process is problematic and can often lead to more or less than the optimum amount of tissue being ablated or removed. Another source of problems is operation, coordination and handling of the multiple (often three or more) separate catheters required simultaneously in the heart during the procedure to provide (1) internal cardiac monitoring and pacing, (2) intracavity mapping (using a multielectrode mapping catheter), (3) ablation, and (4) if required, but not uniformly employed due to its inherent inaccuracy, an ultrasound catheter in the heart. These multiple devices take up valuable space inside the catheter and inside the body of a mammal being treated and the presence of multiple catheters in the heart at one time increases the risk of embolism and stroke due to potential formation of clots or dislodgement of tissue from the heart or vascular wall.

The above description of the background art is not intended to limit the application of the assembly, system and method as disclosed herein.

### SUMMARY OF THE DISCLOSURE

According to a first aspect there is disclosed an optical fibre assembly adapted to be received inside a catheter for ablating a tissue portion of a mammal, the assembly comprising:
(i) a plurality of optical cores, each core defining a leading end and a trailing end and being adapted to carry an optical imaging beam; and
(ii) an optical lens arrangement operatively connected to the leading end of the plurality of optical cores for causing divergence of the light beams emitted therefrom;

wherein the optical fibre assembly is adapted to create a field of view by directing a plurality of said optical imaging beams onto the tissue portion and capturing a reflected portion of said beams.

Preferably, the plurality of optical cores is located in a single optical fibre. Thus, the optical fibre assembly may comprise a multi-core fibre. Preferably, the optical fibre assembly comprises at least 4-7, 10, 15, 20, 25, 30, 35 or 40 optical cores.

Preferably, when in use, only a subset of the optical cores carries the optical imaging beam. In this regard, the optical fibre assembly may be operatively connected to an a system or device for generating an optical imaging beam such as an optical coherence tomography (OCT) system that controls how many beams are produced and hence how many optical cores carry such beams.

Preferably, the plurality of optical cores has a diameter of about 0.5-3 mm, 0.6-2.9 mm, 0.7-2.8 mm, 0.8-2.7 or 0.8-2.5. In this regard, the relatively small form factor of the optical fibre assembly makes it more suitable for insertion into a catheter for insertion into the body of a mammal. The smaller the optical fibre assembly, the less space it occupies in the catheter, leaving more room for other devices or instruments.

The optical cores in the plurality of optical cores may be arranged in a circular pattern. Preferably, the optical cores in the plurality of optical cores are arranged in a circular pattern with at least one optical core located inside the circular pattern, such as in the centre of the circular pattern.

The plurality of optical cores may also be located in separate optical fibres. Thus, the optical fibre assembly may comprise multiple fibres. Preferably, the optical fibre assembly comprises at least 4-7, 10, 15, 20, 25, 30, 35 or 40 optical fibres. Preferably, when in use, only a subset of the optical fibres carries the optical imaging beam.

Preferably, the optical fibre(s) are formed as a fibre optic patch cord including connectors that enable the fibres to be conveniently connected to other devices or components.

Preferably, the divergence of a light beam caused by the optical lens arrangement is at least 20-60, 30-50, 35-45 or 40 degrees relative to a path of propagation of the light beam through a corresponding core.

Preferably, the optical lens arrangement causes the beams to diverge to differing amounts. For example, the optical lens arrangement may cause at least one beam to diverge by a substantial amount and other beams to diverge to a lesser amount. In another example, the optical lens arrangement may cause at least one beam not to diverge at all. It will be appreciated that by forming the optical lens arrangement to suit requirements a desirable field of view can be created for a range of end user applications and situations.

Preferably, the optical lens arrangement comprises a gradient index (GRIN) lens.

Preferably, the optical lens arrangement comprises a gradient index lens and a convex, concave or angular lens.

When the optical lens arrangement comprises a GRIN lens and another lens, the lens may be provided integrally as a single lens. In this regard, the convex, concave or angular lens may be machined or otherwise formed into a leading end of the gradient index lens.

Alternatively, the lens may be separate components affixed or attached together by resin or some other suitable adherent.

Preferably, the optical lens arrangement has a focussing distance of about 0.5-5 mm, 1-5 mm or 2-5 mm.

Preferably, the optical lens arrangement has a diameter of about 0.5-3 mm, 1-2.5 mm or 1-2 mm.

The optical fibre assembly may further comprise an interface to be located between any two components of the optical fibre assembly. Preferably, the interface is adapted to be located between the plurality of optical cores and the lens.

The interface may be adapted to receive at least a subset of the plurality of optical cores. In this regard, the interface may comprise a platform comprising a series of openings or apertures to receive the optical cores and hence allow the optical imaging beam to be transferred between components of the optical fibre assembly. Preferably, the interface is adapted to receive the leading end of the separate optical fibres. In this regard, the interface may define at least one aperture to receive and retain the separate optical fibres.

Preferably, the interface is formed of silicon.

Preferably, the interface has a generally circular cross-section.

Preferably, the interface is affixed or attached in position by epoxy, resin or some other suitable adherent.

Preferably, the optical beam from each optical core illuminates an area of at least 0.1, 0.25, 0.5, 0.75 or 1 mm².

Preferably, the entire field of view of the optical fibre assembly has an area of at least 0.1 cm².

The optical fibre assembly may be incorporated in an OCT system that also includes an optical ablating beam generator capable of generating an optical ablating beam that is propagated along one of the plurality of optical cores. The one of the plurality of optical cores may be a central core arranged to launch the optical ablating beam into the optical lens arrangement at a location so that the optical ablating beam travels through the lens arrangement without divergence relative to a path of propagation through the central core. Further the OCT system may be arranged to switch the optical ablating beam to propagate through any one of the optical cores that at any instant time is in contact with the tissue portion. The optical ablating beam generator may be in the form of a laser.

The optical ablating beam may have a wavelength lying in the range of 808-980 nm.

The optical fibre assembly may further comprise a sensing component.

Preferably, the sensing component comprises a pressure sensor and/or a temperature sensor.

Preferably, the optical fibre assembly is operatively connected to a system or apparatus for generating an optical imaging beam such as an optical coherence tomography (OCT) system.

Thus, according to a second aspect, there is disclosed an OCT system comprising an optical fibre assembly according to a first aspect.

Preferably, the OCT system is able to generate an optical imaging beam at a wavelength of 700-3000 nm such as 1300 nm or 2000 nm, noting that there may be a small variation on either side of the nominated wavelength.

The optical fibre assembly can form part of an OCT system to be used to visualise mammalian tissue.

Thus, according to a third aspect there is disclosed a method of visualising mammalian tissue comprising the steps of:
(i) inserting an optical fibre assembly according to a first aspect of the disclosure into a catheter inserted in a mammal;

(ii) operating the optical fibre assembly to visualise a portion of the mammalian tissue.

Preferably, the method is carried out in real time.

The method may also be used to visualise ablation of the tissue portion. Preferably, the ablation is also performed using the optical fibre assembly described herein that further comprises an ablation means.

### General

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness. The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The term “optical beam” as used herein relates to a beam of light that carries signals and/or optical power. For example, the optical imaging beam can carry signals that may be used for imaging; the ablating beam can carry optical power that may be used for ablation; and the sensing beam can carry signals that may be used for sensing temperature and/or pressure at or near the leading end of the catheter tip assembly. Each beam may be directed, modulated, or transformed, and still be a beam in the sense that the same, or corresponding, signals and/or optical power are still transmitted. For example, a beam may be optically modified (e.g., optically amplified, or modulated, or shifted to a different optical wavelength), and still carry signals and power that are determined and controlled by the signals and the power before modification, and thus this may be regarded as the same beam herein.

The embodiments described herein may include one or more range of values (e.g. size etc.). A range of values will be understood to include all values within the range, including the values defining the range, and values adjacent to the range which lead to the same or substantially the same outcome as the values immediately adjacent to that value which defines the boundary to the range, provided such an interpretation does not read on the prior art.

In this specification the terms “leading” and “following” for example in the phrases “leading end” and “following end” refer to positions relative to the position of a feature relative to the tissue being treated. “Leading” as used herein refers to a feature or part thereof that is closest or proximal to the tissue whereas “following” refers to a feature or part thereof that is furthest or distal to the tissue.

Other definitions for selected terms used herein may be found within this specification and apply throughout. Unless otherwise defined, all technical terms used herein have the same meaning as commonly understood to one of ordinary skill in the art to which the disclosure belongs.

### BRIEF DESCRIPTION OF DRAWINGS

Notwithstanding any other forms which may fall within the scope of the assembly, system and method as set forth in the Summary, specific embodiments will now be described, by way of example only, with reference to the accompanying drawings, in which:

FIG. 1 is a perspective exploded view of a first embodiment of the disclosed optical fibre assembly incorporating an interface between a plurality of optical cores in a single fibre and an optical lens arrangement;

FIG. 2A is a perspective exploded view of a second embodiment of the disclosed optical fibre assembly incorporating an interface between a plurality of optical fibres and an optical lens arrangement;

FIG. 2B is an end view of the interface in FIG. 2A;

FIG. 2C is a view through cross section A-A in FIG. 2B;

FIG. 2D is a perspective exploded view of an embodiment of the optical fibre assembly similar to that in FIG. 2A but incorporating a plurality of optical fibres in a patch cord arrangement;

FIG. 3A is a perspective exploded view of a third embodiment of the disclosed an optical fibre assembly that is similar to that depicted in FIG. 2A but incorporates an optical lens arrangement with a plurality of lens portions;

FIG. 3B is a cross sectional side view of an optical fibre assembly similar to that in FIG. 1 but including an optical lens arrangement similar to that in FIG. 3A, showing the effect of the optical lens arrangement on the path of the optical imaging beams passing therethrough;

FIG. 4A is a cross sectional side view of a further embodiment of the disclosed optical fibre assembly including an alternate optical lens arrangement and showing the effect of the optical lens arrangement on the path of the optical imaging beams passing therethrough; and

FIG. 4B is a cross sectional side view of yet another embodiment of the disclosed optical fibre assembly including another alternate optical lens arrangement and showing the effect of the optical lens arrangement on the path of the optical imaging beams passing therethrough.

### DESCRIPTION OF SPECIFIC EMBODIMENTS

Described herein are embodiments of the disclosed optical fibre assemblies that may allow for improved tissue imaging, tissue ablation, and temperature and/or pressure sensing using a single catheter in a human or animal body. The system may allow one or more of following modalities (or processes) to be provided using a single catheter: determination of vessel or heart wall proximity, thickness and character (e.g., normal pre burn, oedema post burn), determination of vessel wall contact pressure, sensing a temperature of wall tissue, burning using a focussed laser beam and intra cardiac pacing when in the heart.

The optical fibre assemblies described herein are relatively small and hence can be used in a catheter with other components, as required.

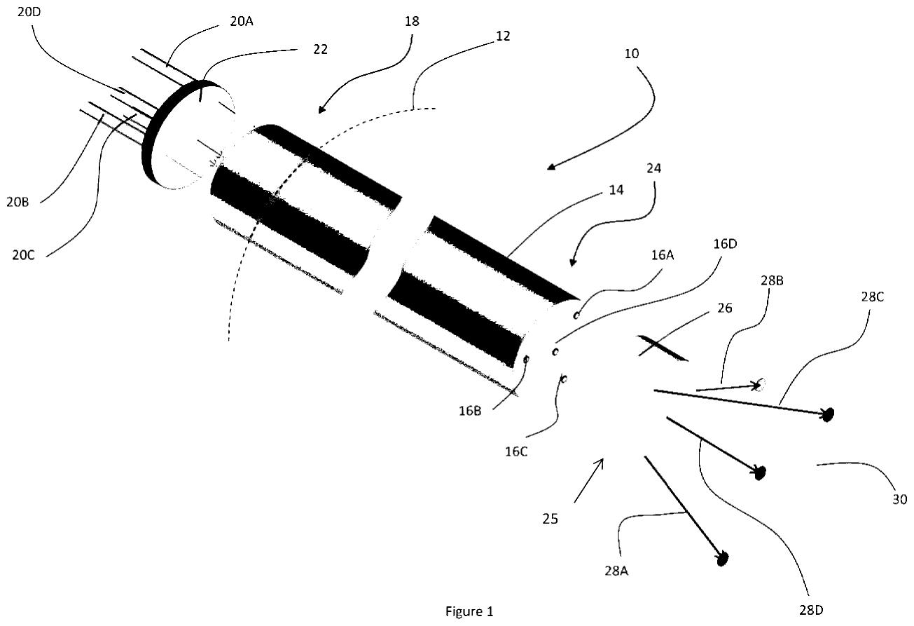

FIG. 1 depicts a first embodiment of the disclosed optical fibre assembly 10, inside a sleeve catheter 12. The assembly 10 includes a plurality of optical cores in the form of a multi-core optical fibre 14, with a diameter of about 1 mm, and including nineteen cores, four of which, 16A-16D, are being utilised in the embodiment to carry optical imaging beams generated by an optical coherence tomography system (not shown) and delivered to the trailing end 18 of the multi-core fibre 14 by four optical fibres 20A-20D.

Optical fibres 20A-20D are operatively connected to cores 16A-16D in the multi-core fibre 14 via an interface in the form of a circular shaped silicon platform 22 that includes four apertures to receive the leading ends of optical fibres 20A-20D and is affixed to the multi-core fibre 14, using a suitable adhesive such as epoxy resin.

Attached to the leading end 24, again by a suitable adhesive such as epoxy resin, is an optical lens arrangement 25 arranged to change the path of light passing there through. In the case of light being transmitted through the lens arrangement, which is incident on the tissue, the change in path causes a divergence of the incident light exiting the lens arrangement 25. In this way the incident light is able to illuminate an area greater than an area of the lens structure 25 transverse to the direction of propagation of light through the optical lens structure 25. In FIG. 1 this is represented by the diverged beams 28A-28C, that correspond to the beams exiting cores 16A-16C, respectively. These beams occupy a field of view 30 which has a larger area than the transverse area of the optical lens arrangement 25.

In this embodiment the propagation path of a central light beam launched or emanating from core 20D that is aligned with the geometric center of the optical lens structure is not altered by passage through the lens arrangement 25.

In one form, the optical lens arrangement 25 may be a graded index (“GRIN”) lens 26. The GRIN lens 26 has flat opposed surfaces.

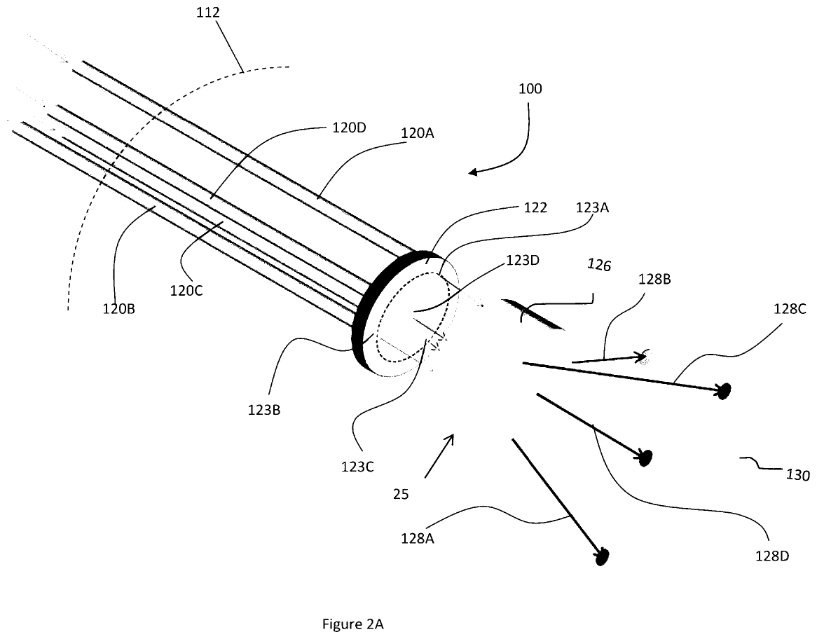

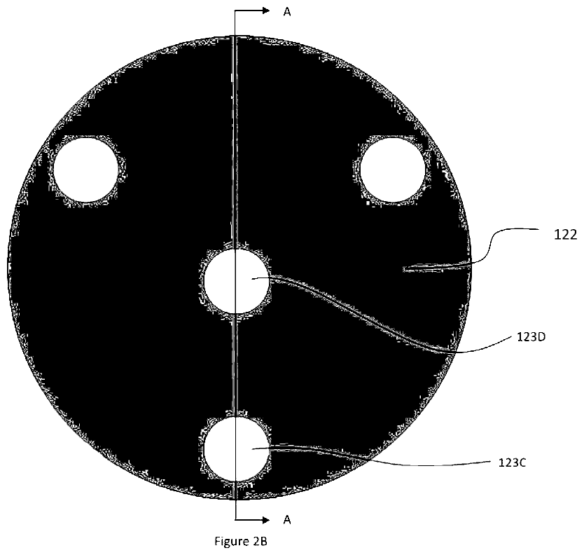

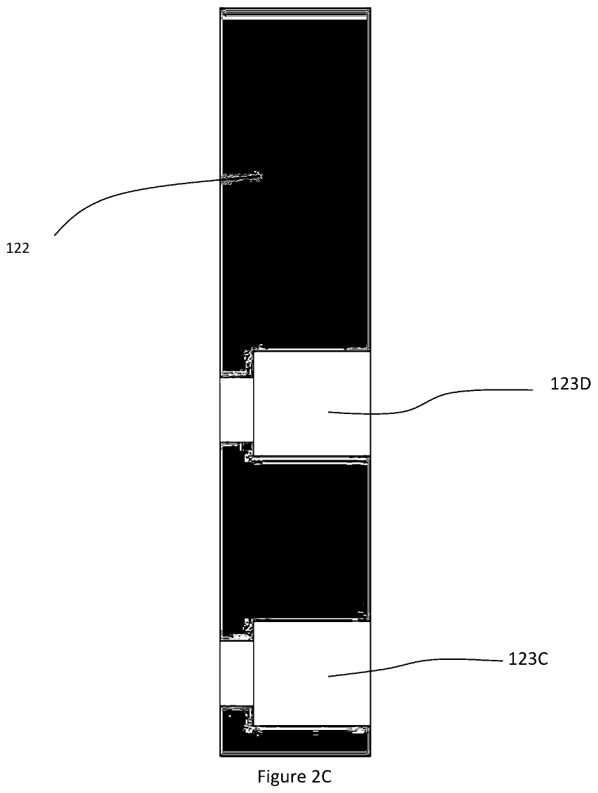

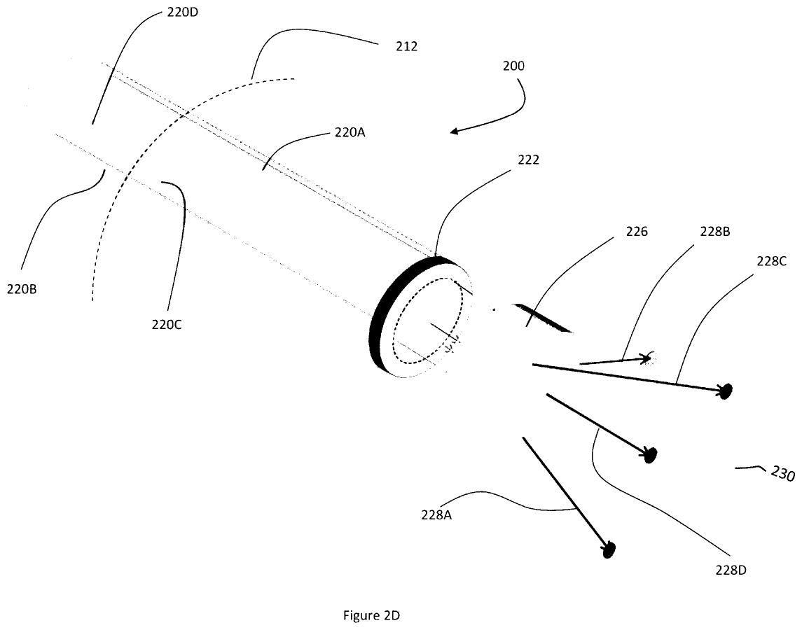

FIGS. 2A-2C depicts a second embodiment of the disclosed optical fibre assembly, generally indicated by the numeral 100 with a diameter of about 0.8-1 mm and being adapted to fit inside a sleeve catheter 112. The assembly 100 includes a plurality of optical cores in the form of four fibres 120A-120D that carry optical imaging beams generated by an optical coherence tomography system (not shown) and are operatively connected to an optical lens arrangement 25 in the form of a GRIN lens 126, via an interface in the form of a circular shaped silicon platform 122 (shown separately in FIG. 2B and in cross section in FIG. 2C) that is the same as that shown in FIG. 1 and includes four apertures 123A-123D, each with a diameter of about 135 μm, to receive the leading ends of optical fibres 120A-120D. The platform 122 is affixed using resin or some other adherent to the GRIN lens 126 that causes divergence of the optical imaging beams exiting the fibres 120A-120C and passing through the GRIN lens 126. The resulting diverged beams 128A-128C, that correspond to the beams exiting fibres 120A-120C, respectively, together form field of view 130.

FIG. 2D depicts a third embodiment of the disclosed optical fibre assembly, generally indicated by the numeral 200 which is according to a third embodiment of the first aspect of the optical fibre assembly, arranged or otherwise adapted to fit inside a sleeve catheter 212. The assembly 200 is similar to assembly 100 in FIG. 2A but includes a plurality of optical cores in the form of four fibres 220A-220D that a provided as a patch cord and carry optical imaging beams generated by an optical coherence tomography system (not shown). The optical fibres 220A-220D are operatively connected to a lens in the form of a GRIN lens 226, via an interface in the form of a circular shaped silicon platform 222 that is the same as that shown in FIG. 1. The platform 222 is affixed using resin or some other adherent to the GRIN lens 226 that causes divergence of the optical imaging beams exiting the fibres 220A-220C and passing through the GRIN lens 226. The resulting diverged beams 228A-228C, that correspond to the beams exiting fibres 220A-220C, respectively, together form field of view 230.

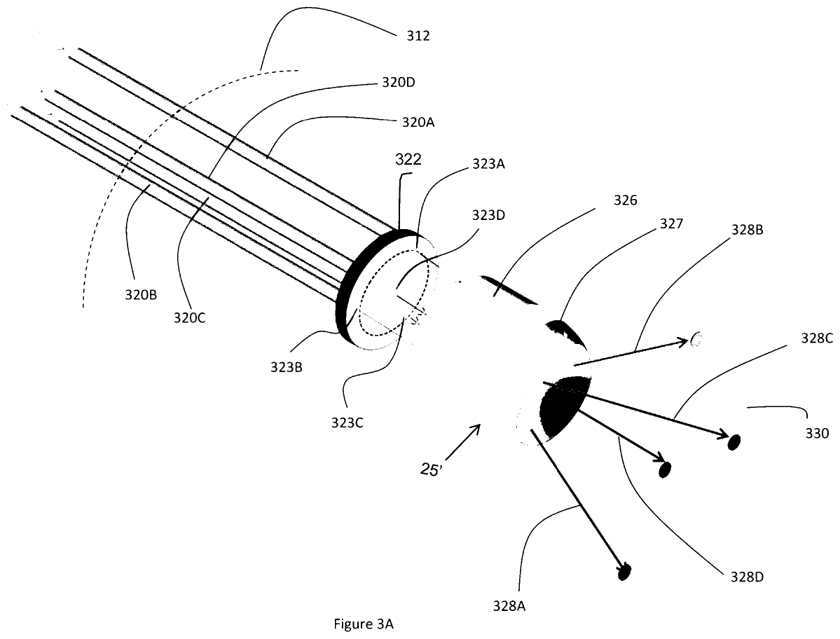

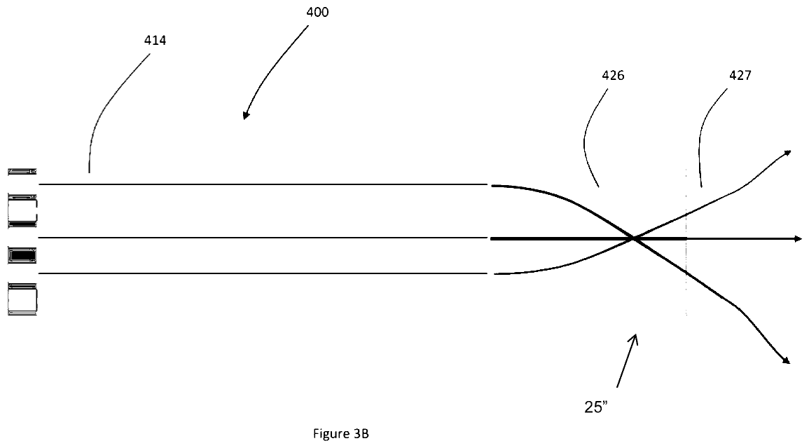

FIG. 3A depicts a fourth embodiment of the disclosed optical fibre assembly, generally indicated by the numeral 300 which is arranged or otherwise adapted to fit inside a sleeve catheter 312. The assembly 300 includes a plurality of optical cores in the form of four fibres 320A-320D that carry optical imaging beams generated by an optical coherence tomography system (not shown) and are operatively connected to an optical lens arrangement 25′ via an interface in the form of a circular shaped silicon platform 322 that is the same as that shown in FIG. 1 and includes four apertures 323A-323D to receive the leading ends of optical fibres 320A-320D. The optical coherence tomography system may be in a form described in the Applicant's international publication number WO 2016/187664 the contents of which is incorporated herein by way of reference.

The lens arrangement 25′ comprises a GRIN lens 326 and a convex lens 327 coupled to a flat surface of the GRIN lens 326 opposite the optical fibres 320A-320D. The platform 322 is affixed using resin or some other adherent to the flat surface GRIN lens 326 adjacent the fibres 320A-320D. The convex lens 327 causes increased divergence of the optical imaging beams exiting the fibres 320A-320C in comparison to passing solely through a GRIN lens 326. The resulting diverged beams 328A-328C, that correspond to the beams exiting fibres 320A-320C, respectively, together form field of view 330.

FIG. 3B is a side view, in cross section, depicting a fifth embodiment of the disclosed optical fibre assembly, generally indicated by the numeral 400. The optical fibre assembly 400 is similar to that shown in FIG. 1, and includes a plurality of optical cores in the form of a multi-core fibre 414. However, it includes a lens arrangement 25″ comprising a GRIN lens 426 combined with a convex lens 427 that causes increased divergence of the optical imaging beams exiting therethrough.

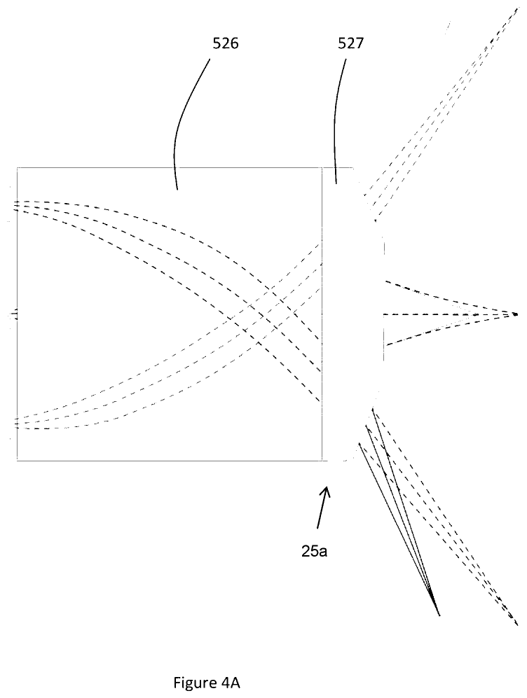

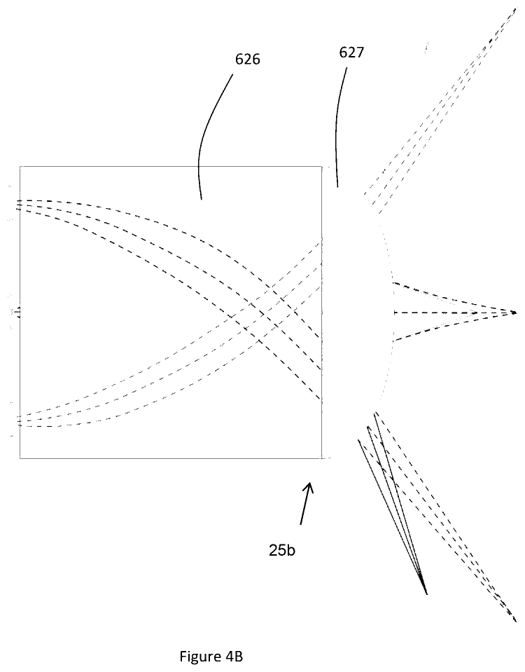

FIGS. 4A and 4B are schematic representations of two further lens arrangements 25a and 25b respectively that can be incorporated in other embodiments of the disclosed optical fibre. The lens arrangement in FIG. 4A comprises a GRIN lens 526 and an angular lens 527. The lens arrangement in FIG. 4B comprises a GRIN lens 626 and a convex lens 627. It will be appreciated that where the lens arrangements comprise two or more parts, the parts may be separate parts that have been joined together or a single structure that has been treated to provide the same effect as the two or more parts, such as by machining or some other surface treatment/shaping process.

Applications

Embodiments of the assembly, system and method may provide effective results when used for procedures, e.g., cardiac ablation. For example in cardiac ablation, this may allow for the combination of the functions of burning, pace making, monitoring, and tissue imaging into a single catheter, thus reducing the number of catheter insertions. In this regard, an embodiment of the disclosed optical fibre assembly has a relatively small form factor that allows it to be used concurrently with other components in a single catheter. Embodiments may allow for more accurate and quicker ablation performance, and may reduce requirements for repeat ablations on the same patient. Embodiments may reduce the total cost of catheters required for an example procedure.

When the optical fibre assembly includes an optical beam as the ablating beam, it may be more accurate and less damaging than using radio frequency (RF) ablation provided by currently existing medical ablation systems, due to more accurate control of width, depth, position and intensity of the burn.

The data captured using the optical fibre assembly can be used to determine ablation intensity and ablation duration e.g., based on the observed tissue depth of the facing tissue portion.

The foregoing is illustrative of the disclosed assembly, system and method and is not to be construed as limiting thereof. Although a number of exemplary embodiments have been described, it should be appreciated that the assembly, system and method may be embodied in many other forms.

In the claims which follow, and in the preceding description, except where the context requires otherwise due to express language or necessary implication, the word “comprise” and variations such as “comprises” or “comprising” are used in an inclusive sense, i.e. to specify the presence of the stated features but not to preclude the presence or addition of further features in various embodiments of the assembly, system and method as disclosed herein.

1. An optical fibre assembly adapted to be received inside a catheter for ablating a tissue portion of a mammal, the assembly comprising:
(i) a plurality of optical cores, each core defining a leading end and a trailing end and being adapted to carry an optical imaging beam; and
(ii) an optical lens arrangement operatively connected to the leading end of the plurality of optical cores for causing divergence of the light beams emitted therefrom;
wherein the optical fibre assembly is adapted to create a field of view by directing a plurality of said optical imaging beams onto the tissue portion and capturing a reflected portion of said beams.

2. An optical fibre assembly according to claim 1 wherein the plurality of optical cores are located in a single optical fibre.

3. (canceled)

4. An optical fibre assembly according to claim 1 wherein the optical fibre assembly has a diameter of about 0.5-3 mm.

5. (canceled)

6. An optical fibre assembly according to claim 1 wherein the plurality of optical cores include at least one core located centrally relative to the other optical cores.

7. An optical fibre assembly according to claim 1 wherein the plurality of optical cores are located in separate optical fibres.

8. (canceled)

9. An optical fibre assembly according to claim 1 wherein the divergence of the beams caused by the lens is at least 20-60 degrees relative to corresponding paths of propagation of the beams through respective cores.

10. An optical fibre assembly according to claim 1 wherein the optical lens arrangements causes at least two beams to diverge by differing amounts.

11. An optical fibre assembly according claim 1 wherein the optical lens arrangement is a lens.

12. An optical fibre assembly according to claim 11 wherein the lens is a gradient index (GRIN) lens or a gradient index lens and at least one of a concave, convex or angular lens.

13. (canceled)

14. An optical fibre assembly according to claim 11 wherein the lens has a focusing distance of about 0.5-5 mm.

15. An optical fibre assembly according to claim 11 wherein the lens has a diameter of about 0.5-3 mm.

16. An optical fibre assembly according to claim 1 comprising an interface located between the plurality of optical cores and the optical lens arrangement.

17. An optical fibre assembly according to claim 16 wherein the interface is adapted to receive at least a subset of the plurality of optical cores.

18. An optical fibre assembly according to claim 16 wherein the interface has a generally circular cross-section.

19. An optical fibre assembly according to claim 1 wherein the field of view has an area of at least 0.1-1 mm².

20. An OCT system comprising an optical fibre assembly according to claim 1 and an optical ablating beam generator capable of generating an optical ablating beam that is propagated along one of the plurality of optical cores.

21. An OCT system according to claim 20 wherein the optical ablating beam generator the one of the plurality of optical cores is a central core arranged to launch the optical ablating beam into the optical lens arrangement at a location so that the optical ablating beam travels through the lens arrangement without divergence relative to a path of propagation through the central core.

22. An OCT system according to claim 20 wherein the optical ablating beam generator is a laser.

23. An OCT system according to claim 20 further arranged to switch the optical ablating beam to propagate through any one of the optical cores that at any instant time is in contact with the tissue portion.

24. A method of visualizing a tissue portion of a body of a mammal in real time comprising the steps of:
(i) inserting an optical fibre assembly according to claim 1 into a catheter inserted in the body; and
(ii) operating the optical fibre assembly to visualize the tissue portion in real time.
